(19) United States
(12) Patent Application Publication
(10) Pub. No.: US 2026/0083593 A1
(43) Pub. Date: Mar. 26, 2026

(54) MALLEABLE EARPLUG ATTACHMENT TO SAFETY GLASSES

(71) Applicant: Keith Hutto, Graniteville, SC (US)

(72) Inventor: Keith Hutto, Graniteville, SC (US)

(21) Appl. No.: 18/891,256

(22) Filed: Sep. 20, 2024

(51) Int. Cl. A61F 9/02; A61F 11/12

(52) U.S. Cl. CPC A61F 9/029; A61F 11/12

(57) ABSTRACT

The present disclosure teaches a pair of safety goggles or glasses with elastic earplug attachments. Wherein, the pair of safety goggles or glasses may be a standard pair of safety goggles or glasses, with no modification or customization required. Wherein, the elastic earplug attachment may include an earplug, an elastic band, and a pocket. Wherein, the elastic band and the pocket may be made as one integrated piece and of an elastic material. Wherein, the size and shape of the pocket may match those of an end of temples of the pair of safety goggles or glasses. Wherein, a user may attach the elastic earplug attachment to the end by encasing the end with the pocket, and detach the elastic earplug attachment from the end by uncovering the end from the pocket. Friction between an inner surface of the pocket and the end may hold the earplug attachment in place.

### TECHNICAL FIELD

The present disclosure relates to the technical field of safety glasses and earplugs.

### BACKGROUND

Safety goggles and earplugs are both commonly used apparatuses for the protection of eyes and hearing respectively in an industrial setting. Earplugs are small in size and can easily be lost or accidentally dropped to the ground, causing not only inconvenience but hygiene and safety concerns. Hence, it would be desirable to find a way to attach earplugs to the temples of a pair of safety goggles. Alternatively, earplugs may also be attached to a regular pair of eyeglasses or sunglasses.

Several methods have been devised to solve the problem, but none is ideal. First, safety goggles are usually designed to last one year or longer, while earplugs are usually designed to be disposable; hence, ideally, the earplugs should be detachably attached to the safety goggles. Second, many state-of-the-art designs require modification or customization of the safety goggles; ideally, the earplugs should be able to be used with a standard pair of safety goggles. Third, many state-of-the-art designs include strings, which could easily tangle with a user's hair or other head-worn apparatuses. Fourth, in some designs, the connections between the earplugs and the temples of the goggles are not sufficiently secure. The presently disclosed technology aims to address these issues and optimize the design of earplug attachments to safety goggles.

### SUMMARY

The presently disclosed technology teaches an apparatus, comprising: a pair of safety goggles or glasses, with two temples, each of the two temples having an end; an earplug attachment detachably attached to each of the two temples, further including: an earplug made of a soundproof material; an elastic band connected to the earplug; a pocket connected to the elastic band; wherein, the pocket and the elastic band are made of an elastic material and as one integrated piece; wherein, the pocket matches the end of each of the two temples; wherein, a user is able to attach the earplug attachment to the end of each of the two temples by encasing the end of each of the two temples with the pocket, and detach the earplug attachment from the end of each of the two temples by uncovering the end of each of the two temples from the pocket; wherein, friction between an inner surface of the pocket and the end of each of the two temples act upon an entirety of the inner surface of the pocket and is able to hold the earplug attachment in place.

In some embodiments, the pair of safety goggles or glasses is a pair of Z87+ safety glasses.

In some embodiments, the inner surface of the pocket is textured.

In some embodiments, the elastic material is rubber.

In some embodiments, the soundproof material is foam.

In some embodiments, the earplug attachment is disposable.

In some embodiments, the pair of safety goggles or glasses requires no modification or customization.

In some embodiments, the end of each of the two temples is textured.

In some embodiments, the elastic band is thin and elongated.

In some embodiments, the elastic band is reinforced by a string in a center of the elastic band.

The presently disclosed technology also teaches an earplug attachment, comprising: an earplug made of a soundproof material; an elastic band connected to the earplug; a pocket connected to the elastic band; wherein, the pocket and the elastic band are made of an elastic material and as one integrated piece; wherein, the pocket matches an end of each of two temples of a pair of safety goggles or glasses; wherein, a user is able to attach the earplug attachment to the end of each of the two temples by encasing the end of each of the two temples with the pocket, and detach the earplug attachment from the end of each of the two temples by uncovering the end of each of the two temples from the pocket; wherein, friction between an inner surface of the pocket and the end of each of the two temples act upon an entirety of the inner surface of the pocket and is able to hold the earplug attachment in place.

In some embodiments, the pair of goggles is a pair of Z87+ safety glasses.

In some embodiments, the inner surface of the pocket is textured.

In some embodiments, the elastic material is rubber.

In some embodiments, the soundproof material is foam.

In some embodiments, the earplug attachment is disposable.

In some embodiments, the pair of safety goggles or glasses requires no modification or customization.

In some embodiments, the end of each of the two temples is textured.

In some embodiments, the elastic band is thin and elongated.

In some embodiments, the elastic band is reinforced by a string in a center of the elastic band.

### BRIEF DESCRIPTION OF THE DRAWINGS

The present disclosure is further illustrated by way of exemplary embodiments, which are described in detail through the accompanying drawings. These embodiments are not limiting, and in these embodiments, the same numbering indicates the same structure, wherein:

FIG. 1 is a structural diagram showing an elastic earplug attachment, according to some embodiments of the presently disclosed technology.

FIG. 2 is a structural diagram showing a pair of safety goggles with the elastic earplug attachments, according to some embodiments of the presently disclosed technology.

FIG. 3 is a structural diagram showing the pair of safety goggles with the elastic earplug attachments in use, according to some embodiments of the presently disclosed technology.

### DETAILED DESCRIPTION

In order to more clearly illustrate the technical solutions of the embodiments of the present disclosure, the accompanying drawings for the description of the embodiments are described below. Obviously, the accompanying drawings in the following description are only some examples or embodiments of the present disclosure, and it is possible for a person of ordinary skill in the art to apply the present disclosure to other similar scenarios in accordance with these accompanying drawings without creative labor. Unless obviously obtained from the context or the context illustrates otherwise, the same numeral in the drawings refers to the same structure or operation.

It should be understood that the terms “system,” “device,” “unit,” and/or “module” are used herein as a way to distinguish between different components, elements, parts, sections, or assemblies at different levels. However, if other words may achieve the same purpose, the terms may be replaced with alternative expressions.

As indicated in the present disclosure and in the claims, unless the context clearly suggests an exception, the words “one,” “a,” “a kind of,” and/or “the” do not refer specifically to the singular but may also include the plural. In general, the terms “include” and “comprise” suggest only the inclusion of clearly identified steps and elements, which do not constitute an exclusive list, and the method or device may also include other steps or elements.

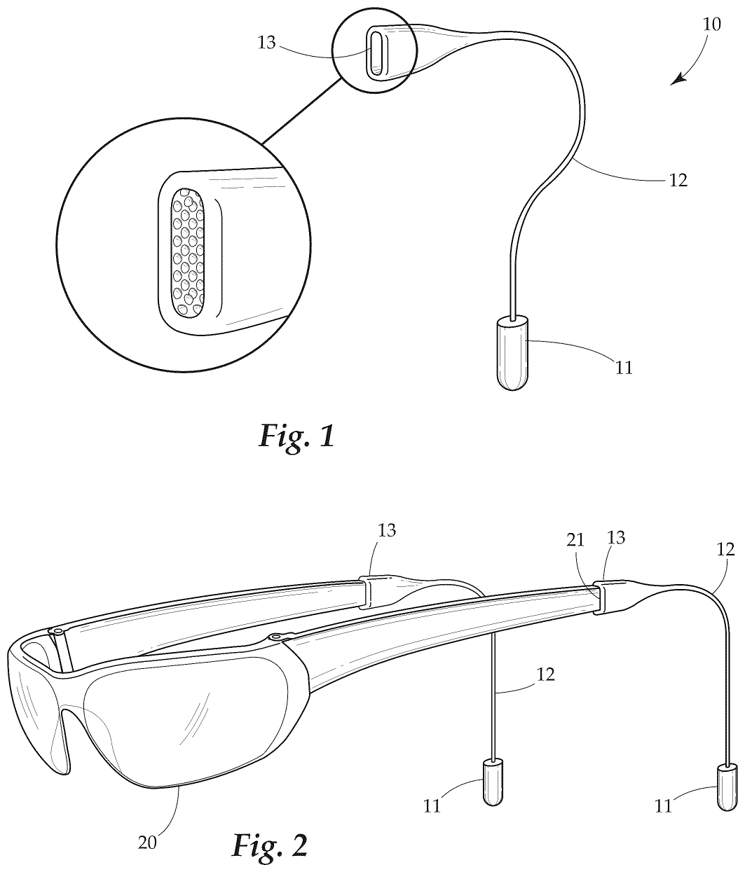

FIG. 1 is a structural diagram showing an elastic earplug attachment, according to some embodiments of the presently disclosed technology.

In some embodiments, the elastic earplug attachment 10 may include an earplug 11, an elastic band 12, and a pocket 13. In some embodiments, the earplug 11 may be the same as or similar to a state-of-the-art earplug made of foam or other soundproofing materials, such as rubber, silicone, acrylic materials, wax with a cotton or felt cover, thermoplastic elastomers, or any combination thereof. Hereafter, the phrase “soundproofing material” may refer to any of the above-mentioned materials, including foam, rubber, silicone, acrylic materials, wax with cotton or felt cover, thermoplastic elastomers, any other materials with good acoustic insulation properties, or any combination thereof. In some embodiments, the earplug 11 may be replaced by a wireless earbud or hearing aid. Alternatively, a wireless earbud or hearing aid may be embedded in the earplug 11. In some embodiments, the wireless earbud may play white noises, safety instructions in an industrial setting, music, podcasts, etc. In some embodiments, operators may communicate via these wireless earbuds in an industrial setting. In some embodiments, the elastic band 12 may be an elongated, thin, and malleable piece connecting the earplug 11 and the pocket 13. In some embodiments, the elastic band 12 may be reinforced by a string in the center of the elastic band.

In some embodiments, the elastic band 12 and the pocket 13 may be made of the same material and manufactured as an integrated piece, for example, in a single molding process. In some embodiments, the back of pocket 13 may be closed and tapered at its end toward the elastic band 12. The integration of the elastic band 12 and the pocket 13 may simplify the manufacturing process, reduce the risk of the connecting part between the elastic band and the pocket tangling with a user's hair or other hand-worn apparatuses, and provide a visual indication when the elastic earplug attachment 10 is properly secured to the end of the temple. In some embodiments, the elastic band 12 and the pocket 13 may be made of rubber or similarly elastic materials. This way, the elastic band is less likely to get entangled with a user's hair or other head-worn apparatuses than a string.

In some embodiments, the earplug 11 may also be made of the same material as the elastic band 12 and the pocket 13, and the entire earplug attachment 10 may be manufactured as an integrated piece. For example, the entire earplug attachment 10 may be made of rubber. This integration could further simplify the manufacturing process of the earplug attachment 10. In some embodiments, In other embodiments, the earplug 11 may be made of a different material as the elastic band 12 and the pocket 13, and/or manufactured as separate pieces. For example, the earplug 11 may be attached to one end of the elastic band 12 using adhesives. For another example, a hole may be pre-designed on the earplug 11, and one end of the elastic band 12 may be inserted into the hole in the manufacturing process and further secured in the hole using adhesives.

The pocket 13 may be detachably attached to the end of a temple of a pair of safety goggles. In some embodiments, the shape of the pocket 13 may be customized according to the shape of the ends of the temples of the goggles in use. The shape of the pocket 13 may match the shape of the end of the temple so that a friction between an inner surface of the pocket 13 and the end of the temple of the goggles may hold the elastic earplug attachment 10 in place. Given the elasticity of the pocket 13, the friction attachment surface between the pocket and the end of the temple could be the entire inner surface of the pocket, so the security of such friction attachment may be guaranteed. If the pocket 13 is made of a rigid material, the friction attachment between the pocket and the end of the temple may only occur at a few connecting points, making such friction attachment less secure. In some embodiments, the inner surface of the pocket 13 may have certain textures to increase the friction between the inner surface of the pocket and the end of the temple of the goggles so that the connection between the pocket and the end of the temple may be more secure. For example, the inner surface of pocket 13 may be embossed or patterned to include ridges or bumps. For another example, techniques such as sandblasting may be used to roughen the inner surface of the pocket 13, to give it a more matte or tactile feel. As discussed above, the pocket 13 may be made of rubber or other materials with high elasticities.

As discussed in the background section, in some embodiments, the elastic earplug attachment 10 is designed to be disposable for hygiene reasons. The elastic earplug attachment 10 may be detachably attached to a standard pair of safety goggles that may be designed to last a long period of time. As discussed in the background section, the presently disclosed elastic earplug attachment may be attached to a standard pair of safety goggles without requiring any modification or customization to the safety goggles.

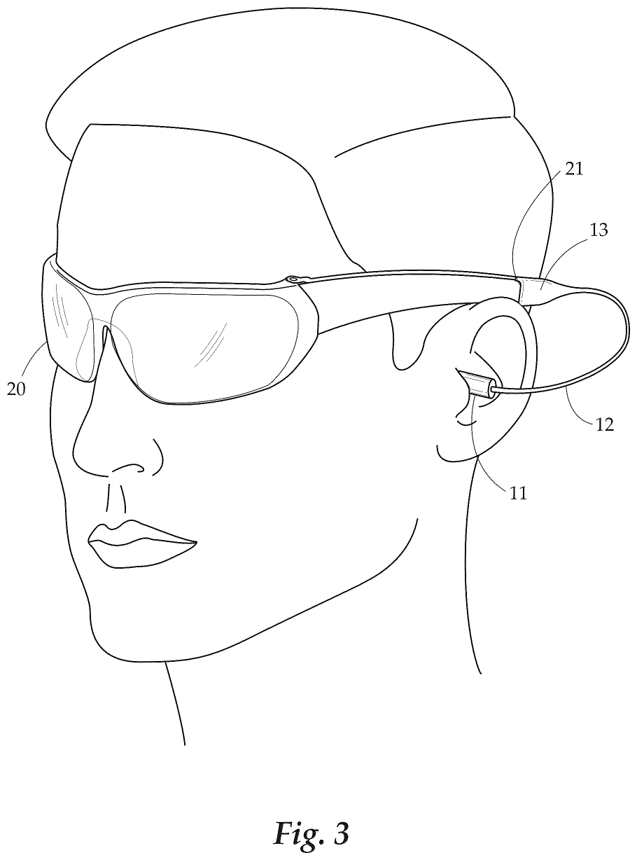

FIG. 2 is a structural diagram showing a pair of safety goggles with the elastic earplug attachments, according to some embodiments of the presently disclosed technology. FIG. 3 is a structural diagram showing the pair of safety goggles with the elastic earplug attachments in use, according to some embodiments of the presently disclosed technology.

In some embodiments, the presently disclosed technology may include a standard pair of safety goggles 20. In some embodiments, the pair of safety goggles may be Z87+ safety glasses. In some embodiments, the elastic earplug attachments 10 may be detachably attached to the ends of temples 21 of the pair of safety goggles 20 without requiring any modification or customization to the standard pair of safety goggles. As discussed above, in some embodiments, the size and shape of the pocket 13 may match the size and shape of the end of a temple 21 of a pair of Z87+ safety glasses. In some embodiments, the size and shape of the pocket 13 may match the size and shape of the end of a temple 21 of other models of safety goggles 20 in use. In some embodiments, a user may attach the pocket 13 to the end of temple 21 by encasing the end of the temple with the pocket and detach the pocket from the end of the temple by uncovering the end of the temple from the pocket. In some embodiments, the ends of temples 21 of the safety goggles 20 may have textured surfaces so that the friction attachment between the inner side of the pocket 13 and the end of the temple of the goggles may be more secure. For example, the ends of the temples 21 may be embossed or patterned with small ridges or bumps. For another example, the ends of the temples 21 may be sandblasted. For yet another example, stickers with an adhesive back surface and a frosted front surface may be placed on the ends of the temples 21. In some embodiments, pocket 13 may have extra high elasticity so that it could fit onto safety goggles 20 with ends of the temples 21 of different shapes and sizes. In some embodiments, the end of temple 21 may include a notch or a marking to provide a visual indication that the end of the temple has been inserted into the pocket 13 fully and properly.

In some embodiments, the lenses of the safety goggles 20 are made from impact-resistant materials like polycarbonate or acetate. The lenses may be with or without prescriptions. In some embodiments, the lenses may be cut and shaped according to the mold specifications and treated for scratch resistance and UV protection. In some embodiments, the frame of the safety goggles 20 may be made from durable materials like rubber, silicone, or thermoplastic and molded. In some embodiments, the lenses may be inserted into the goggles. In some embodiments, the safety goggles 20 may include additional parts such as adjustable straps or ventilation systems.

In some embodiments, a regular pair of eyeglasses or sunglasses may be used in the presently disclosed technology instead of the safety goggles.

Furthermore, unless explicitly stated in the claims, the use of order, numbers, letters, or other names for processing elements and sequences are not intended to limit the order of the processes and methods of the present disclosure. While various examples have been discussed in the disclosure as currently considered useful embodiments of the invention, it should be understood that such details are provided for illustrative purposes only. The appended claims are not limited to the disclosed embodiments, and instead, the claims are intended to cover all modifications and equivalent combinations within the scope and essence of the embodiments disclosed in the present disclosure. For example, although the described system components may be implemented through a hardware device, they may also be realized solely through a software solution, such as installing the described system on an existing processing or mobile device.

Similarly, it should be noted that, for the sake of simplifying the presentation of embodiments disclosed in the present disclosure and aiding in understanding one or more embodiments of the present disclosure, various features have been sometimes combined into a single embodiment, drawing, or description. However, this manner of disclosure does not imply that the features required by the claims are more than the features mentioned in the claims. In fact, the features of the embodiments are less than all the features of the single embodiment disclosed in the foregoing disclosure.

In some embodiments, numeric values describing the composition and quantity of attributes are used in the description. It should be understood that such numeric values used for describing embodiments may be modified with qualifying terms such as “about,” “approximately” or “generally”. Unless otherwise stated, “about,” “approximately” or “generally” indicates that a variation of ±20% is permitted in the described numbers. Accordingly, in some embodiments, the numerical parameters used in the disclosure and claims are approximations, which can change depending on the desired characteristics of the individual embodiment. In some embodiments, the numerical parameters should take into account a specified number of valid digits and employ a general manner of bit retention. Although the numerical ranges and parameters used in some embodiments of the present disclosure to confirm the breadth of the range are approximations, in specific embodiments, such numerical values are set as precisely as practicable.

With respect to each of the patents, patent applications, publications of patent applications, and other material, such as articles, books, specifications, publications, documents and the like, cited in the present disclosure, the entire contents thereof are hereby incorporated herein by reference. Application history documents that are inconsistent with the contents of the present disclosure or that create conflicts are excluded, as are documents (currently or hereafter appended to the present disclosure) that limit the broadest scope of the claims of the present disclosure. It should be noted that in the event of any inconsistency or conflict between the descriptions, definitions, and/or use of terminology in the materials appended to the present disclosure and the contents described herein, the descriptions, definitions, and/or use of terminology in the present disclosure shall prevail.

In closing, it should be understood that the embodiments described in the present disclosure are used only to illustrate the principles of the embodiments of the present disclosure. Other deformations may also fall within the scope of the present disclosure. Therefore, by way of example and not limitation, alternative configurations of the embodiments disclosed in the present disclosure may be considered consistent with the teachings of the present disclosure. Accordingly, the embodiments described in the present disclosure are not limited to the explicitly introduced and described embodiments in the present disclosure.

What is claimed is:

1. An apparatus, comprising:
a pair of safety goggles or glasses, with two temples, each of the two temples having an end;
an earplug attachment detachably attached to each of the two temples, further including:
an earplug made of a soundproof material;
an elastic band connected to the earplug;
a pocket connected to the elastic band;
wherein, the pocket and the elastic band are made of an elastic material and as one integrated piece;
wherein, the pocket matches the end of each of the two temples;
wherein, a user is able to attach the earplug attachment to the end of each of the two temples by encasing the end of each of the two temples with the pocket, and detach the earplug attachment from the end of each of the two temples by uncovering the end of each of the two temples from the pocket;
wherein, friction between an inner surface of the pocket and the end of each of the two temples act upon an entirety of the inner surface of the pocket and is able to hold the earplug attachment in place.

2. The apparatus as in claim 1, wherein the pair of safety goggles or glasses is a pair of Z87+ safety glasses.

3. The apparatus as in claim 1, wherein the inner surface of the pocket is textured.

4. The apparatus as in claim 1, wherein the elastic material is rubber.

5. The apparatus as in claim 1, wherein the soundproof material is foam.

6. The apparatus as in claim 1, wherein the earplug attachment is disposable.

7. The apparatus as in claim 1, wherein the pair of safety goggles or glasses requires no modification or customization.

8. The apparatus as in claim 1, wherein the end of each of the two temples is textured.

9. The apparatus as in claim 1, wherein the elastic band is thin and elongated.

10. The apparatus as in claim 1, wherein the elastic band is reinforced by a string in a center of the elastic band.

11. An earplug attachment, comprising:
an earplug made of a soundproof material;
an elastic band connected to the earplug;
a pocket connected to the elastic band;
wherein, the pocket and the elastic band are made of an elastic material and as one integrated piece;
wherein, the pocket matches an end of each of two temples of a pair of safety goggles or glasses;
wherein, a user is able to attach the earplug attachment to the end of each of the two temples by encasing the end of each of the two temples with the pocket, and detach the earplug attachment from the end of each of the two temples by uncovering the end of each of the two temples from the pocket;
wherein, friction between an inner surface of the pocket and the end of each of the two temples act upon an entirety of the inner surface of the pocket and is able to hold the earplug attachment in place.

12. The earplug attachment as in claim 11, wherein the pair of goggles is a pair of Z87+ safety glasses.

13. The earplug attachment as in claim 11, wherein the inner surface of the pocket is textured.

14. The earplug attachment as in claim 11, wherein the elastic material is rubber.

15. The earplug attachment as in claim 11, wherein the soundproof material is foam.

16. The earplug attachment as in claim 11, wherein the earplug attachment is disposable.

17. The earplug attachment as in claim 11, wherein the pair of safety goggles or glasses requires no modification or customization.

18. The earplug attachment as in claim 11, wherein the end of each of the two temples is textured.

19. The apparatus as in claim 11, wherein the elastic band is thin and elongated.

20. The apparatus as in claim 11, wherein the elastic band is reinforced by a string in a center of the elastic band.
